(19) United States
(12) Patent Application Publication
(10) Pub. No.: US 2008/0153839 A1
(43) Pub. Date: Jun. 26, 2008

(54) USE OF ANISIC ACID FOR PROMOTING DESQUAMATION

(75) Inventors: Thierry Cotton, Paris (FR); David Amar, Paris (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 11/954,416

(22) Filed: Dec. 12, 2007

Related U.S. Application Data
(60) Provisional application No. 60/870,850, filed on Dec. 20, 2006.

(30) Foreign Application Priority Data
Dec. 12, 2006 (FR) 06 55445

(51) Int. Cl. A61K 31/192; A61K 31/495; A61K 31/426; A61Q 19/00

(52) U.S. Cl. 514/252.12; 514/568; 514/369

(57) ABSTRACT

Use of anisic acid or a derivative thereof for promoting desquamation of the skin and/or for stimulating epidermal renewal. Associated compositions. Especially useful for improving the radiance of the complexion and/or the grain of the skin and/or for smoothing out the skin's microrelief, and/or for attenuating age spots.

### REFERENCE TO PRIOR APPLICATIONS

This application claims priority to U.S. provisional application 60/870,850 filed Dec. 20, 2006, and to French patent application 0655445 filed Dec. 12, 2006, both incorporated herein by reference.

### FIELD OF THE INVENTION

The present invention relates to the use of a composition comprising, in a physiologically acceptable medium, at least one of anisic acid and derivatives of anisic acid, for promoting desquamation of the skin and/or for stimulating epidermal renewal.

The invention also relates to the use of at least one of anisic acid and derivatives of anisic acid in a composition containing a physiologically acceptable medium, as an agent for promoting desquamation of the skin and/or for stimulating epidermal renewal.

The invention is especially intended for improving the radiance of the complexion and/or the grain of the skin and/or for smoothing the skin's microrelief, and/or for attenuating age spots.

Additional aspects and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Desquamation is a natural phenomenon associated with the fact that the epidermis, which constitutes the upper layer of the skin and of the scalp, is in constant regeneration.

The epidermis contains several layers of cells, the deepest of which is the basal layer consisting of undifferentiated cells. Over time, these cells differentiate and migrate towards the surface of the epidermis, forming the various layers thereof, until they form at the surface of the epidermis the corneocytes, which are dead cells that become removed by desquamation. This surface loss is compensated for by the migration of cells from the basal layer to the surface of the epidermis. This constitutes a perpetual renewal of the skin. Forced removal of the horny layer accelerates the renewal and makes it possible to combat ageing.

At the same time, these cells continue their differentiation, the final stage of which is the corneocyte. Corneocytes are in effect dead cells that constitute the last layer of the epidermis, i.e. the outermost layer also known as the stratum corneum.

It is known that the desquamation process may be impaired by exogenous factors (e.g.: UV radiation, pollution, allergens or pathogens) and/or endogenous factors (e.g.: hormonal changes, age, etc.) and especially lead to a slowing-down of epidermal renewal and consequently to ageing of the skin and/or thickening of the horny layer (e.g.: formation of calluses).

Impairments in the desquamation process may also lead to desquamative disorders of aesthetic type (e.g.: dandruff, squamae, etc.) or of pathological type (e.g.: xerosis, ichthyosis, psoriasis or atopic dermatitis).

As more particularly regards ageing of the skin, resulting from intrinsic or extrinsic factors, it is generally reflected by a change in the appearance of the skin, which may be manifested, for example, by: dry skin, the appearance of squamae, a complexion of cloudy, dull and/or yellow appearance, skin of rough and/or cracked appearance, the appearance of wrinkles and fine lines, the appearance of age spots, and/or increased visibility of the pores.

### SUMMARY OF THE INVENTION

There is thus a need to find a way to promote desquamation and/or stimulate the epidermal renewal of the skin. The inventors have done this, and have demonstrated that anisic acid, which has been known hitherto as a microbial agent, especially in hair compositions for treating the scalp (EP 1 325 731) or in deodorant compositions (WO 87/06827), also has exfoliant properties. Specifically, they have shown that anisic acid, applied topically to a model of surviving skin, reduces the cohesion of the stratum corneum and thus promotes the process of desquamation and of epidermal renewal of the skin.

### DETAILED DESCRIPTION OF THE PREFERRED EMBODIMENTS

For the purposes of the present invention, the term “anisic acid derivative” means a salt of anisic acid or a C₁₋₄ alkyl ester of anisic acid. In a preferred embodiment, it is an alkali metal or alkaline-earth metal salt, an ammonium salt or a salt with an organic amine, or alternatively a C₁₋₄ alkyl ester. These derivatives do not include hydroxy derivatives of methoxybenzoic acid, such as those described in patent applications FR 2 739 556, US 2002/028 254 and U.S. Pat. No. 5,766,613.

As used herein, the two phrases “anisic acid or a derivative thereof” and “at least one of anisic acid and derivatives of anisic acid” each mean one or more than one compound selected from the group consisting of anisic acid and derivatives of anisic acid.

The invention compositions may especially be skincare compositions and/or skin cleansing compositions.

The term “skin” is used in the broad sense to include skin and semi-mucous membranes (lips).

The invention compositions are especially intended for caring for and/or cleansing the skin, in particular for caring for and/or cleansing aged skin, or skin whose complexion has a dull and/or rough appearance.

The present invention thus relates in part to the use of a composition comprising, in a physiologically acceptable medium, anisic acid or a derivative thereof, for promoting desquamation of the skin and/or for stimulating epidermal renewal.

The invention also relates to the use of anisic acid or a derivative thereof in a composition containing a physiologically acceptable medium, as an agent for promoting desquamation of the skin and/or for stimulating epidermal renewal.

In other words, the anisic acid or a derivative thereof or the composition containing it is intended according to the invention for promoting desquamation of the skin and/or for stimulating epidermal renewal.

Anisic acid (or a derivative thereof) per se and/or the composition containing it are especially intended according to the invention for improving at least one of the conditions chosen from:
improving the radiance and/or homogeneity of the complexion and/or reducing the cloudy and/or dull appearance of the complexion;
improving the surface appearance, in particular reducing the rough or cracked appearance of the skin and/or improving the grain and/or softness of the skin;
smoothing out the skin's microrelief, in particular smoothing out the wrinkles and fine lines and/or attenuating acne or chickenpox marks;
attenuating age spots (e.g.: senile lentigo) and/or aesthetic pigmentation disorders, such as brownish pigmentation spots and/or freckles;
reducing the dryness of the skin, in particular the dryness of aged skin;
improving the appearance and/or reducing the visibility of the pores and/or improving the unblocking of the pores;
promoting the cleansing action and the removal of dead cells at the surface of the skin; and/or
combating the imperfections of greasy skin, in particular the shiny or lustrous appearance of the skin.

Anisic acid (or a derivative thereof) per se and/or the composition containing it may also be used for the preparation of compositions for treating pigmentation disorders, such as melasmas, post-inflammatory hyperpigmentation and accidental hyperpigmentations, possibly due to photosensitization or post-lesional scarring.

The invention thus relates to the use of a composition containing, in a physiologically acceptable medium, anisic acid or a derivative thereof, or to the use of anisic acid or a derivative thereof per se for improving the radiance and/or homogeneity of the complexion and/or for reducing the cloudy and/or dull appearance of the complexion.

The invention is also directed towards the use of a composition comprising, in a physiologically acceptable medium, anisic acid or a derivative thereof, or the use of anisic acid or a derivative thereof per se for improving the surface appearance, in particular for reducing the rough or cracked appearance of the skin and/or for improving the grain and/or softness of the skin.

The invention also relates to the use of a composition containing, in a physiologically acceptable medium, anisic acid or a derivative thereof, or to the use of anisic acid or a derivative thereof per se, for smoothing out the skin's microrelief, in particular for smoothing out wrinkles and fine lines and/or for attenuating acne or chickenpox marks.

The invention also relates to the use of a composition containing, in a physiologically acceptable medium, anisic acid or a derivative thereof, or to the use of anisic acid or a derivative thereof per se, for attenuating age spots.

The invention is also directed towards the use of a composition containing, in a physiologically acceptable medium, anisic acid or a derivative thereof, or the use of anisic acid or a derivative thereof per se, for reducing dryness of the skin, in particular the dryness of aged skin.

Also included in the context of the invention is the use of a composition containing, in a physiologically acceptable medium, anisic acid or a derivative thereof, or the use of anisic acid or a derivative thereof per se, for improving the appearance and/or reducing the visibility of the pores and/or improving the unblocking of the pores.

The invention also covers the use of a composition containing, in a physiologically acceptable medium, anisic acid or a derivative thereof, or the use of anisic acid or a derivative thereof per se, for promoting the cleansing action and the removal of dead cells at the surface of the skin.

The invention also relates to the use of a composition containing, in a physiologically acceptable medium, anisic acid or a derivative thereof, or to the use of anisic acid or a derivative thereof per se, for combating imperfections of greasy skin, in particular the shiny or lustrous appearance of the skin.

The invention method and composition is preferably used by subjects desirous of the benefits noted above, subjects “in need of” these benefits. Such subjects are typically suffering from skin issues, such as detected by self diagnosis or cosmetician or medical diagnosis, or are at recognized and appreciated risk of developing such conditions and who use the invention methods and compositions to combat these effects. In this regard, the invention process can be viewed as one for correcting various skin issues as described above and/or for delaying the onset of the appearance of, and/or for reducing signs of, skin issues as described above.

Naturally, one using the invention as disclosed will use the compositions described herein to cover the area of the skin being treated in a single application, and of course includes repeated application, for example on a daily basis over a course of days, weeks, etc. In a preferred embodiment the invention process includes multiple applications of the invention composition to the area(s) of skin in need of attention.

### Anisic Acid and Derivatives

The anisic acid used in the present invention may be used in its native form or may be in the form of an alkali metal or alkaline-earth metal salt, an ammonium salt or a salt with an organic amine, or alternatively in the form of a C₁₋₄ alkyl ester. The alkyl group of the ester may be linear or branched, and examples that may be mentioned include methyl, ethyl, n-propyl, isopropyl, n-butyl and tert-butyl groups.

Examples of anisic acid salts or esters that may especially be mentioned include sodium anisate, potassium anisate, methyl anisate, ethyl anisate, propyl anisate and butyl anisate.

Preferably, the anisic acid is used in its ortho-, meta- or para-form, also known as ortho-, meta- or para-methoxybenzoic acid, and even more preferentially para-anisic acid or 4-methoxybenzoic acid.

In particular, para-anisic acid or 4-methoxybenzoic acid or para-methoxybenzoic acid sold by the company Merck may be used.

For the purposes of the present invention, the term “anisic acid derivative” means a salt of anisic acid or a C₁₋₄ alkyl ester of anisic acid.

In particular, it will be an alkali metal or alkaline-earth metal salt, an ammonium salt or a salt with an organic amine, or alternatively a C₁₋₄ alkyl ester.

These derivatives do not include hydroxy derivatives of methoxybenzoic acid, such as those described in patent applications FR 2 739 556, US 2002/028 254 and U.S. Pat. No. 5,766,613.

The anisic acid or a derivative thereof is especially used in an amount to obtain the desired desquamating effect. By way of example, the amount of anisic acid or derivative thereof in the composition may range from 0.001% to 50% by weight relative to the total weight of the composition, preferably from 0.01% to 5% by weight relative to the total weight of the composition and even more preferentially from 0.05% to 2% by weight relative to the total weight of the composition. More preferably, a composition containing from 0.1% to 0.5% by weight of anisic acid or a derivative thereof relative to the total weight of the composition will be used.

According to one particular mode of the invention, concerning scrubbing compositions, anisic acid or a derivative thereof may be used in the compositions in an amount ranging from 1% to 50% by weight relative to the total weight of the composition, preferably from 1% to 30% by weight and even more preferentially from 5% to 20% by weight relative to the total weight of the composition.

The compositions containing anisic acid according to the invention may be applied to any area of the skin or its appendages, especially of the face, the body, the neckline or the hands, or to the lips.

According to one particular mode of the invention, the composition is intended for caring for and/or cleansing aged skin.

According to another mode, the composition is intended for caring for and/or cleansing skin with a dull and/or rough appearance.

According to yet another mode, the composition is intended for caring for and/or cleansing greasy skin.

### Galenics

The compositions according to the invention preferably comprise a physiologically acceptable medium. The term “physiologically acceptable medium” denotes a medium that is compatible with human skin and semi-mucous membranes (lips).

This physiologically acceptable medium comprises water, optionally mixed with one or more organic solvents such as C₁-C₈ alcohols, especially ethanol, isopropanol, tert-butanol or n-butanol, polyols, for instance glycerol, propylene glycol, butylene glycol and sorbitol, and polyol ethers.

The compositions according to the invention may be cosmetic or dermatological compositions and may thus comprise a cosmetically or pharmaceutically acceptable medium. Preferably, such a composition will be a cosmetic composition intended especially for caring for and/or cleansing the skin.

The composition may also comprise a fatty phase, which may comprise oils, gums or waxes usually used in the field of application under consideration. As oils or waxes that may be used in the invention, mention may be made of mineral oils (liquid petroleum jelly), plant oils (liquid fraction of shea butter, sunflower oil, apricot oil, rice bran oil, etc.), animal oils (perhydrosqualene), synthetic oils (Purcellin oil, isononyl isononanoate or pentaerythrityl tetraethylhexanoate), silicone oils or waxes (cyclomethicone, dimethicone or phenyl methicone), fluoro oils (perfluoropolyethers), silicone gums (cyclopentasiloxane and dimethiconol), silicone polymers (of the type such as KSG or dimethicone vinyl dimethicone crosspolymer), beeswax, carnauba wax or paraffin wax, shea butter or hydrogenated jojoba oil. Fatty alcohols (cetyl alcohol, stearyl alcohol, etc.), fatty acids (stearic acid, etc.) or consistency factors such as the mixture of ethylene glycol acetyl stearate and glyceryl tristearate (Unitwix®) may be added to these oils.

When the composition is an emulsion, the proportion of the fatty phase may range from 5% to 80% by weight and preferably from 5% to 50% by weight relative to the total weight of the composition. The oils, waxes, emulsifiers and co-emulsifiers used in the composition in emulsion form are chosen from those conventionally used in cosmetics. The emulsifier and the co-emulsifier are present in the composition in a proportion ranging from 0.3% to 30% by weight and preferably from 0.5% to 20% by weight relative to the total weight of the composition. The emulsion may also contain lipid vesicles.

When the composition is an oily solution or gel, the fatty phase may represent more than 90% of the total weight of the composition.

The composition may also contain adjuvants that are common in the field under consideration, such as surfactants, emulsifiers, gelling agents, hydrophilic or lipophilic active agents, preserving agents, antioxidants, solvents, fragrances, fillers, tensioning agents (natural and synthetic), UV-screening agents, odour absorbers and dyestuffs, and other cosmetic or pharmaceutical active agents.

The amounts of these various adjuvants are those that are conventionally used in cosmetics, for example from 0.01% to 10% of the total weight of the composition. Depending on their nature, these adjuvants may be introduced into the fatty phase, into the aqueous phase and/or into lipid spherules.

As surfactants that may be used, examples that may be mentioned include glyceryl stearate, polysorbate-60 and the mixture of PEG-6/PEG-32/glycol stearate sold under the name Tefose® 63 by the company Gattefossé; PEG-stearate derivatives, sugar derivatives, sucrose esters and phosphate surfactants (e.g.: Amphisol®).

As hydrophilic gelling agents that may be used in the invention, mention may be made of carboxyvinyl polymers (carbomer), acrylic copolymers such as acrylate/alkyl-acrylate copolymers, polyacrylamides, crosslinked or non-crosslinked 2-acrylamido-2-methylpropanesulfonic acid (AMPS) copolymers, polysaccharides such as hydroxypropylcellulose, natural gums and clays, and lipophilic gelling agents that may be mentioned include modified clays, for instance bentones, metal salts of fatty acids, for instance aluminium stearate, hydrophobic silica, ethylcellulose and polyethylene.

As UV-screening agents or photoprotective agents active in the UVA and/or UVB range that may be used in the composition of the invention, mention may be made especially of organic or mineral photoprotective agents.

The organic photoprotective agents are especially chosen from anthranilates; cinnamic derivatives; dibenzoylmethane derivatives; salicylic derivatives; camphor derivatives; triazine derivatives such as those described in patent applications U.S. Pat. No. 4,367,390, EP 863 145, EP 517 104, EP 570 838, EP 796 851, EP 775 698, EP 878 469, EP 933 376, EP 507 691, EP 507 692, EP 790 243 and EP 944 624; benzophenone derivatives; β,β-diphenylacrylate derivatives; benzotriazole derivatives; benzalmalonate derivatives; benzimidazole derivatives; imidazolines; bis-benzazolyl derivatives as described in patents EP 669 323 and U.S. Pat. No. 2,463,264; p-aminobenzoic acid (PABA) derivatives; methylenebis(hydroxyphenylbenzotriazole) derivatives as described in patent applications U.S. Pat. No. 5,237,071, U.S. Pat. No. 5,166,355, GB 2 303 549, DE 197 26 184 and EP 893 119; and screening polymers and screening silicones such as those described especially in patent application WO 93/04665; α-alkylstyrene-based dimers such as those described in patent application DE 198 55 649.

The mineral photoprotective agents are chosen from pigments or nanopigments (mean size of the primary particles: generally between 5 nm and 100 nm and preferably between 10 nm and 50 nm) of coated or uncoated metal oxides, for instance titanium oxide (amorphous or crystallized in rutile and/or anatase form), iron oxide, zinc oxide, zirconium oxide or cerium oxide nanopigments, which are all U-photo-protective agents that are well known per se. Standard coating agents are, moreover, alumina and/or aluminium stearate. Such coated or uncoated metal oxide nanopigments are described in particular in patent applications EP 518 772 and EP 518 773.

The photoprotective agents are generally present in the composition according to the invention in proportions ranging from 0.1% to 20% by weight relative to the total weight of the composition and preferably ranging from 0.2% to 15% by weight relative to the total weight of the composition.

The composition may be in any conceivable galenical form suitable for application to the skin.

The composition may especially be in the form of an aqueous, alcoholic, aqueous-alcoholic or oily solution; a dispersion of the lotion or serum type; a multi-phase (e.g.: two-phase) lotion, a water-in-oil, oil-in-water or multiple emulsion; a suspension; microcapsules or microparticles; vesicular dispersions of ionic and/or nonionic type; in the form of a serum; a mousse, a solid preparation, for example a stick; an aerosol composition also comprising a pressurized propellant or in the form of a patch, which may itself be in various galenical forms.

In the family of patches, mention may be made especially of:
controlled-release patches with a hydrophobic polymer matrix as described, for example, in document FR 2 738 744;
patches of reservoir type containing a reservoir of active substance, a diffusion membrane and an adhesive layer;
patches with a magnetic field effect promoting the penetration of the active agents into the skin, as described, for example, in FR 2 791 750;
patches of optimized adhesion, comprising a layer of hydrophobic polymer attached to a support layer and containing particles of active compound, oil particles and particles of a water-absorbing agent as described, for example, in FR 2 761 889;
gel patches with a high water content, of hydrogel type, especially based on at least one hydrocolloid and/or on polysaccharides, proteins, polyacrylamide, polyacrylic acid, acrylic acid and starch copolymers, polyvinylpyrrolidone or polyvinylic acid derivatives, and having a contact adhesion similar to that resulting from a suction cup effect;
hydrophobic gel patches, also known as organogels or oleogels, based on polyethylene, silicones or styrene and isoprene copolymer or styrene and butadiene copolymer. These systems have the advantage of being able to contain large proportions of hydrophobic compounds;
segmented polyurethane-based gel patches (SPUG) whose lipophilic or hydrophilic nature may be modified by modifying the polyoxyethylenated chains (Y. Shikinami, Adhesive polymer gels for medical uses, Kagaku To Tokyo, 67 (4), 141-150, 1993);
leaves or pastilles described in FR 2 512 651 or FR 2 538 247;
composite films as described in EP-A-0 285 563 or US 2002/0 187 181; advantageously, a composite film comprising a reservoir layer constituted by a matrix formed from a silicone polymer inside which are arranged inclusions constituted by the active aqueous phase gelled by means of at least one gelling agent, may be used (FR 2 650 747 L'Oreal);
solid fin films based on natural or synthetic polymers which, when applied to a premoistened nail, dissolve in situ; such films are described in patent applications WO 02/053197, WO 02/05789, WO 2004/032859 and WO 02/054997;
films containing a film-forming agent and a hydrophilic adhesive polymer, also known as patch-non-patch, as described in patent WO 02/30402;
polymer-based compositions with adhesive properties capable of forming a film that is removed after drying (EP-0 514 760, EP-0 826 364) or an elastic, flexible solid layer that is removed by applying a tissue or an adhesive plastic (WO 93/05893); or gels or pastes which, after application, dry to give a film that is removed by washing, cleansing or peeling; these will generally be referred to as “varnish-patches” or “peel-patches”.

It is also possible, however, to use systems or devices that increase the absorption of the anisic acid or a derivative thereof contained in the composition via an active mechanism (M. R. Prausnitz et al., Current status and future potential of transdermal drug delivery, Nature Rev., vol. 3, 115-124, 2004), for instance:
systems for injection under pressure or without a needle (Burkoth T. L. et al., Transdermal and transmucosal powdered drug delivery., Crit Rev Ther Drug Carrier Syst. 1999; 16(4):331-84;
the use of ultrasound: sonophoresis and phonophoresis (Joshi A. and Raje J., Sonicated transdermal drug transport, J. Control. Release, 83 (1), 13-22, 2002);
the use of electrical currents: iontophoresis, electroporation, microfrequency current (Riviere J. E. and Heit M. C., Electrically-assisted transdermal drug delivery, Pharm. Res. 14 (6), 687-697, 1997) (patents U.S. Pat. No. 6,148,232, WO 03/035167);
the use of magnetic waves: magnetophoresis (Murthy S. N., Magnetophoresis: an approach to enhance transdermal drug diffusion., Pharmazie. 1999 May; 54(5):377-9);
the use of microneedles (McAllister D. V. et al., Microfabricated microneedles for gene and drug delivery, Annu. Rev. Biomed. Eng., 2, 289-313, 2000) (U.S. Pat. No. 6,451,240).

The composition according to the invention may be in the form of a cleansing or makeup-removal composition, an antisun composition or a treatment or care composition for the face, for the hands, for the feet, for the major anatomical folds or for the body. By way of example, the composition may be a day cream, a night cream, a makeup-removing cream, an SPF composition for protecting against sunlight, a protective or care body milk, an after-sun milk, a skincare lotion, gel or mousse, a cleansing lotion, an artificial tanning composition or a shaving cream or mousse.

When the composition according to the invention is intended for cleansing and/or removing makeup from the skin, it may also be in the form of a solution or a foaming gel or a foaming cream with or without soap or a makeup-removing oil.

According to this particular mode, the composition according to the invention will preferably comprise at least one surfactant, which will give the composition the cleansing or even foaming nature. This surfactant may be chosen from nonionic surfactants, anionic surfactants, cationic surfactants, amphoteric and zwitterionic surfactants, and mixtures thereof.

The surfactant may be present in a composition according to the invention in an amount ranging from 0.1% to 50% by weight, preferably from 0.5% to 20% by weight, in particular from 1% to 15% by weight or even from 5% to 10% by weight relative to the total weight of the composition.
a) The nonionic surfactants may be chosen, for example, from alkyl polyglucosides (APG), maltose esters, polyglycerolated fatty alcohols, glucamine derivatives, for instance 2-ethylhexyloxycarbonyl-N-methylglucamine, and mixtures thereof.
b) The anionic surfactants may be chosen, for example, from soaps (alkali metal salts of fatty acids), carboxylates, acylamino acids, amidoether carboxylates, alkyl polyaminocarboxylates, alkyl ether sulfates such as sodium laureth sulfates, alkyl sulfonates, isethionates, alkyl methyltaurates, alkyl sulfosuccinates, alkylsulfoacetates, alkyl phosphates (monoalkyl or dialkyl phosphates), salts thereof, and mixtures thereof.
c) The amphoteric and zwitterionic foaming surfactants may be chosen, for example, from betaine derivatives, including amidopropylbetaines, amphoacetates and amphodiacetates, and hydroxylsultaines, and mixtures thereof.

According to one embodiment of the invention, a surfactant that is suitable for the invention may be chosen from alkylpolyglucosides, betaine derivatives, alkyl glycol carboxylic acids and salts thereof, alkyl ether sulfates, alkyl phosphates, amphodiacetates, amphoacetates, alkylglycinates, acyl glutamates, acyl sarcosinates and mixtures thereof.

According to one particular mode, a surfactant that is suitable for the invention may be chosen from decyl glucoside, cocoyl glucoside, sodium lauryl ether sulfate, cocoyl betaine, lauroyl betaine, cocamidopropyl betaine, lauramidopropyl betaine, lauryl glycol carboxylate, cocoampho(di)acetate, lauroampho(di)acetate and potassium lauryl phosphate, and a mixture thereof.

Preferred surfactants that may especially be mentioned include fatty acids; polysorbates (e.g.: polysorbate 85); PEG-stearates and isostearates such as PEG-20 glyceryl stearate; sodium N-cocoylglycinate; and oxyethylenated behenyl alcohol.

When the composition according to the invention is intended for a use of scrubbing type, it may also be in any galenical form mentioned above provided that it is easily removed by rinsing, and especially in the form of an aqueous gel or an aqueous or aqueous-alcoholic solution. It may be applied via any means allowing uniform distribution and especially using cotton wool, a cotton bud, a brush, a gauze, a spatula or a pad, or alternatively by spraying, and may be removed by rinsing with water or using a mild detergent. According to one preferred embodiment of the invention, the composition intended for chemical scrubbing contains a continuous aqueous phase.

According to one advantageous mode of the invention, the desquamating properties of the anisic acid may be combined with other skincare cosmetic agents.

In particular, the composition according to the invention may also comprise at least one other cosmetic active agent chosen from other desquamating agents, moisturizers, antioxidants, depigmenting agents, agents for stimulating the energy metabolism of cells, agents for stimulating epidermal proliferation or differentiation and/or for synthesis of epidermal or dermal macromolecules, agents for reducing or inhibiting the activity of harmful proteases, agents for promoting the skin's barrier function, tensioning agents, calmatives and anti-seborrhoeic agents, and mixtures thereof.

According to a first mode, the composition according to the invention will comprise, besides anisic acid or a salt thereof, at least one other desquamating agent.

According to another alternative, the composition according to the invention will comprise, besides anisic acid or a salt thereof, at least one antioxidant.

According to another mode, the composition according to the invention will comprise, besides anisic acid or a salt thereof, at least one depigmenting agent.

According to another mode, the composition according to the invention will comprise, besides anisic acid or a salt thereof, at least one agent for stimulating the energy metabolism of cells.

According to another mode, the composition according to the invention will comprise, besides anisic acid or a salt thereof, at least one agent for stimulating epidermal proliferation or differentiation and/or the synthesis of epidermal or dermal macromolecules.

According to another mode, the composition according to the invention will comprise, besides anisic acid or a salt thereof, at least one agent for reducing or inhibiting the activity of harmful proteases.

According to another mode, the composition according to the invention will comprise, besides anisic acid or a salt thereof, at least one agent for promoting the skin's barrier function.

According to another mode, the composition according to the invention will comprise, besides anisic acid or a salt thereof, at least one tensioning agent.

According to one alternative, the composition according to the invention will comprise, besides anisic acid or a salt thereof, at least one anti-seborrhoeic agent.

The composition may also comprise a moisturizer. According to another mode, it may also comprise a calmative.

Examples of such agents are mentioned below.

Mention may be made especially of:

1) other desquamating agents, such as:
α-hydroxy acids, such as glycolic acid, citric acid, lactic acid, tartaric acid, malic acid or mandelic acid; gentisic acid and derivatives thereof; cinnamic acid; jasmonic acid and derivatives thereof, as in patent applications EP 1 333 022 and EP 1 333 021;
β-hydroxy acids, in particular salicylic acid and derivatives thereof, including 5-n-octanoylsalicylic acid known under the INCI name: Capryloyl salicylic Acid;
(N-2-hydroxyethylpiperazine-N-2-ethane)sulfonic acid (HEPES); 2-oxothiazolidine-4-carboxylic acid (procysteine) derivatives; derivatives of α-amino acids of glycine type (as described in EP-0 852 949, and also the sodium methyl glycine diacetate sold by BASF under the trade name Trilon M®); (3-hydroxy-2-pentylcyclopentyl)acetic acid; azelaic acid; nicotinic acid and derivatives thereof; ascorbic acid and derivatives thereof, such as ascorbyl glucoside and magnesium ascorbyl phosphate;
urea and derivatives thereof, such as hydroxyalkyl derivatives thereof and in particular the N-(2-hydroxyethyl)urea available commercially, in the form of a mixture at 50% by weight in water, from the company National Starch under the trade name Hydrovance®;
8-hexadecene-1,16-dicarboxylic acid or 9-octadecenedioic acid also known as dioic acid, and derivatives thereof; dioic acid is especially commercially available from the company Uniqema under the trade name Arlatone Dioic DCA®.

The use of anisic acid in combination with other desquamating agents advantageously makes it possible to reduce the amount of desquamating agents that is effective to obtain the desired effect.

2) moisturizers, which means:
either a compound that acts on the barrier function, in order to maintain the moisturization of the stratum corneum, or an occlusive compound. Mention may be made of ceramides, sphingoid-based compounds, lecithins, glycosphingolipids, phospholipids, cholesterol and derivatives thereof, phytosterols (stigmasterol, β-sitosterol, campesterol), essential fatty acids, 1,2-diacylglycerol, 4-chromanone, pentacyclic triterpenes such as ursolic acid, petroleum jelly and lanolin;
or a compound that directly increases the water content of the stratum corneum, such as threalose and derivatives thereof, hyaluronic acid and derivatives thereof, glycerol, pentanediol, sodium pidolate, serine, xylitol, sodium lactate, polyglyceryl acrylate, ectoin and derivatives thereof, chitosan, oligosaccharides and polysaccharides, cyclic carbonates, N-lauroylpyrrolidonecarboxylic acid and N-α-benzoyl-L-arginine; C-glycoside derivatives such as those described in EP 1 345 919, in particular C-β-D-xylopyranoside-2-hydroxypropane sold by the company Chimex under the name Mexoryl SBB® (INCI name: hydroxypropyl tetrahydropyrantriol); an extract of Laminaria ochroleuca such as Laminaine® from Secma, which stimulates the synthesis of glycosaminoglycans;
or a compound that activates the sebaceous glands, such as steroid derivatives (including DHEA, 7-oxido and/or 17-alkyl derivatives thereof and sapogenins), methyl dihydrojasmonate, and vitamin D and derivatives thereof.
   3) antioxidants or free-radical scavengers, such as polyphenols; vitamin E and derivatives thereof, in particular vitamin E acetate; vitamin A and esters thereof, in particular vitamin A palmitate; chelating agents such as EDTA; wheatgerm extracts with superoxide dismutase activity, phytanetriol, lignans, idebenone, vitamin C, coenzyme Q10, ellagic acid and biotin.
   4) depigmenting agents, such as vitamin C and derivatives thereof and especially vitamin CG, CP and 3-O-ethyl vitamin C, alpha and beta arbutin, lucinol and derivatives thereof, kojic acid, resorcinol and derivatives thereof, tranexamic acid and derivatives thereof, gentisic acid, homogentisate, methyl gentisate or homogentisate, ellagic acid and vitamin B3.
   5) agents that stimulate the energy metabolism of cells, such as biotin, an extract of Saccharomyces cerevisiae such as Phosphovital® from Sederma, the mixture of sodium, manganese, zinc and magnesium salts of pyrrolidonecarboxylic acid, for instance Physiogenyl® from Solabia, a mixture of zinc, copper and magnesium gluconate such as Sepitonic M3® from SEPPIC, and mixtures thereof.
   6) agents that stimulate epidermal proliferation or differentiation and/or the synthesis of dermal or epidermal macromolecules, such as:
agents that stimulate keratinocytes proliferation, especially including retinoids such as retinol and esters thereof, including retinyl palmitate; adenosine; phloroglucinol; the nut cake extracts marketed by the company Gattefossé; and extracts of Solanum tuberosum sold by the company Sederma;
agents that stimulate keratinocytes differentiation, for instance minerals such as calcium; a peptide extract of lupin, such as the product sold by the company Silab under the trade name Structurine®; sodium beta-sitosteryl sulfate such as the product sold by the company Seporga under the trade name Phytocohesine®; and a water-soluble extract of corn such as the product sold by the company Solabia under the trade name Phytovityl®; the extract of Fagus sylvatica beech buds sold by the company Gattefossé under the trade name Gatuline® RC; a peptide extract of Voandzeia substerranea, such as the product sold by the company Laboratoires Sérobiologiques under the trade name Filladyn LS 9397®; and lignans such as secoisolarici-resinol;
agents that stimulate fibroblast proliferation, for instance plant proteins or polypeptides, especially extracted from soybean (for example a soybean extract sold by the company LSN under the name Eleseryl SH-VEG 8® or sold by the company Silab under the trade name Raffermine®); and plant hormones such as giberrellins and cytokinins;
agents that stimulate collagen synthesis, such as a protein hydrolysate of soybean sold by Silab under the name Nutripeptides, extracts of Centella asiatica, especially madecassoside, asiaticosides and derivatives; synthetic peptides such as iamin, the biopeptide CL or palmitoyloligopeptide sold by the company Sederma; peptides extracted from plants, such as the soybean hydrolysate sold by the company Coletica under the trade name Phytokine®; and plant hormones such as auxins and lignans;
   7) agents that reduce or inhibit the activity of harmful proteases, such as agents that act:
either on the inhibition of metalloproteases (matrix metalloproteases or MMP) such as, more particularly, MMP 1, 2, 3 and 9. Mention may be made of: retinoids and derivatives, oligopeptides and lipopeptides, lipoamino acids, the malt extract sold by the company Coletica under the trade name Collalift®; extracts of mulberry or of rosemary; lycopene; isoflavones, derivatives thereof or plant extracts containing them, in particular extracts of soybean (sold, for example, by the company Ichimaru Pharcos under the trade name Flavosterone SB®), of red clover (sold, for example, by the company Sederma under the name Sterocare®), of flax, of kakkon or of sage; extracts of Cucurma longa; extracts of Siegesbeckia (sold, for example, by the company Sederma);
or on the inhibition of certain serine proteases such as leukocyte elastase or cathepsin G. Mention may be made of: the peptide extract of leguminous seeds (Pisum sativum) sold by Laboratoires Sérobiologiques under the trade name Parelastyl®; heparinoids; an extract of rice peptides such as Colhibine® from Pentapharm; and pseudodipeptides such as {2-[acetyl-(3-trifluoromethylphenyl)amino]-3-methylbutyrylamino}acetic acid.
   8) agents that promote the skin's barrier function, such as an extract of Thermus thermophilus such as Venuceane® from Sederma, an extract of the rhizome of wild yam (Dioscorea villosa) such as Actigen Y® from Active Organics, plankton extracts, for instance Omega Plankton® from Secma, yeast extracts, for instance Relipidium® from Coletica, a chestnut extract such as Recoverine® from Silab, a cedar bud extract such as Gatuline Zen® from Gattefossé, sphingosines, for instance salicyloyl sphingosine sold under the name Phytosphingosine® SLC by the company Degussa, a mixture of xylitol, polyxylityl glycoside and xylitan, for instance Aquaxyl® from SEPPIC, extracts of Solanacea plants, for instance Lipidessence® from Coletica; hydroxyapatite, in particular Hydroxysomes® from the company LSC, and mixtures thereof.
   9) tensioning agents, for instance plant proteins, in particular soybean proteins (e.g.: Eleseryl sold by Silab), colloidal particles of mineral filler, in particular colloidal silica particles, and synthetic polymers, in particular latices.
   10) calmatives, for instance β-glycyrrhetinic acid and salts or derivatives thereof (stearyl glycyrrhetate, 3-stearoyloxyglycyrrhetic acid, glycyrrhetinic acid monoglucuronide) and also plants containing them (e.g.: Glycyrrhiza glabra); ursolic acid and salts thereof; extracts of Centella asiatica, canola oil, bisabolol; camomile extracts, allantoin; a mixture of waterlily blossom extract and of palmitoylproline, such as the product sold under the name Seppicalm VG® by the company SEPPIC; Aloe vera, rosewater, an extract of mint, in particular of mint leaves, for instance Calmiskin® from Silab, filamentous bacteria, for instance Vitreoscilla filiformis as described in patent EP 761 204, an extract of rose petals, for instance Rose Flower Herbasol® extract from the company Cosmetochem, shea butter, and mixtures thereof;
   11) anti-seborrhoeic agents especially such as:
zinc salts such as zinc gluconate, zinc pyrrolidone carboxylate (or zinc pidolate), zinc lactate, zinc aspartate, zinc carboxylate, zinc salicylate or zinc cysteate; and preferably zinc pyrrolidone carboxylate (or zinc pidolate) or zinc salicylate; potassium alum;
clove extract, such as the product sold under the name Clove Extract Powder by the company Maruzen;
glycine grafted onto an undecylenic chain, such as the product sold under the name Lipacide UG OR by the company SEPPIC;
tri(C₁₂-C₁₃)alkyl citrate sold under the name Cosmacol® ECI by the company Sasol; tri(C₁₄-C₁₅)alkyl citrate sold under the name Cosmacol® ECL by the company Sasol.

The additional active agent(s) is (are) generally present in the cosmetic compositions in a content ranging from 0.001% to 10% by weight relative to the total weight of the composition, preferably from 0.01% to 5% and even more preferentially from 0.1% to 2% by weight relative to the total weight of the composition.

The invention also relates to a cosmetic composition for caring for and/or cleansing the skin, comprising, in a physiologically acceptable medium, (i) at least anisic acid or a derivative thereof and (ii) from 0.001% to 10% by weight, relative to the total weight of the composition, of at least one desquamating agent other than citric acid.

Examples of desquamating agents that may be used in the composition according to the invention are described above.

In particular, the desquamating agent is chosen from α-hydroxy acids, β-hydroxy acids, (N-2-hydroxyethylpiperazine-N-2-ethane)sulfonic acid (HEPES); 2-oxothiazolidine-4-carboxylic acid (procysteine) derivatives; urea and derivatives thereof, dioic acid and derivatives thereof.

A preferred desquamating agent is 5-n-octanoylsalicylic acid.

The anisic acid or a derivative thereof will preferably be present in the composition in a content ranging from 0.001% to 50% by weight relative to the total weight of the composition, preferably from 0.01% to 5% by weight relative to the total weight of the composition and even more preferentially from 0.05% to 2% by weight relative to the total weight of the composition.

For scrubbing applications, the composition according to the invention may comprise contents of anisic acid or of a derivative thereof ranging for example from 1% to 50% by weight relative to the total weight of the composition, preferably from 1% to 30% by weight and even more preferentially from 5% to 20% by weight relative to the total weight of the composition.

According to one preferred mode, the composition according to the invention also comprises, in addition to anisic acid and the other desquamating agent, at least one cosmetic active agent chosen from moisturizers, antioxidants, depigmenting agents, agents for stimulating the energy metabolism of cells, agents for stimulating epidermal proliferation or differentiation or for stimulating the synthesis of epidermal or dermal macromolecules, agents for reducing or inhibiting the activity of harmful proteases, agents for promoting the skin's barrier function, tensioning agents, calmatives and anti-seborrhoeic agents.

Examples of such additional agents are described previously in the description.

Preferred additional agents that may especially be mentioned include: urea and derivatives thereof, in particular the N-(2-hydroxyethyl)urea sold commercially, in the form of a mixture at 50% by weight in water, from the company National Starch under the trade name Hydrovance®, ceramides; C-glycoside derivatives such as those described in EP 1 345 919, in particular C-β-D-xylopyranoside-2-hydroxypropane sold by the company Chimex under the name Mexoryl SBB®; tocopherols; adenosine; soybean proteins; rice proteins; a malt extract; a beech bud extract; a corn extract; ellagic acid, ascorbic acid and derivatives thereof, biotin, hydroxyapatite, allantoin, and a mint extract.

In particular, the composition according to the invention will contain at least 2, preferably at least 3 and even more preferentially at least 4 different additional agents.

These additional agents are generally present in the composition in a content ranging from 0.001% to 10% by weight relative to the total weight of the composition.

The care and/or cleansing compositions according to the invention containing anisic acid will be advantageous for caring for and treating all skin types.

In particular, the compositions will be used for caring for and/or cleansing aged skin, skin with a dull appearance, skin with a rough appearance, dry skin and greasy skin, or for caring for and treating individuals having skin of this type.

The invention also relates to a treatment process for promoting desquamation and/or for stimulating epidermal renewal, comprising the application to the skin of a composition comprising, in a physiologically acceptable medium, at least anisic acid or a derivative thereof.

The invention also relates to a treatment process for promoting the radiance of the complexion and/or for reducing skin surface irregularities, comprising the application to the skin of a composition comprising, in a physiologically acceptable medium, at least anisic acid or a derivative thereof.

The term “surface irregularities” especially means the visible and/or tactile irregularities at the surface of the skin such as age spots, wrinkles and fine lines, the increased visibility of the pores, the rough appearance of the skin, or acne or chickenpox marks.

According to one particular mode, a composition as defined above, comprising at least anisic acid and at least one other desquamating agent, is applied to the skin.

According to one particular mode, the composition is applied to aged skin.

The term “aged skin” means skin that shows signs of cutaneous ageing such as wrinkles and fine lines, and/or a loss of elasticity and/or of suppleness and/or of firmness, especially on the face.

These skin types especially characterize people over 25 years old and in particular over 30 years old. The term “mature skin” will be used for people at least 40 years old, and “very mature skin” will be used for people at least 50 or even at least 60 years old.

According to another mode, the composition is applied to skin with a dull and/or rough appearance.

According to yet another mode, the composition is applied to greasy skin.

Finally, the use of anisic acid or a derivative thereof according to the present invention finds another application in the field of chemical scrubbing.

Scrubbing is a well-known means for improving the appearance and/or texture of the skin and/or the scalp, especially for improving the radiance and homogeneity of the complexion and/or for reducing the visible and/or tactile irregularities of the skin, and in particular for improving the surface appearance of the skin, for attenuating actinic lentigo, acne or chickenpox marks, and also for preventing, attenuating or combating the signs of ageing of the skin, and especially for smoothing out skin texture irregularities, such as wrinkles and fine lines.

They have the effect of removing a superficial part of the skin to be treated (epidermis and possibly upper layer of the dermis) via chemical methods.

Also therefore included in the invention is a process for treating the visible and/or tactile irregularities of human skin, comprising:
a) topically applying to the skin a composition containing anisic acid or a derivative thereof;
b) leaving the composition in contact with the skin for a time of between 5 minutes and 6 hours and preferably between 5 minutes and 30 minutes, and
c) removing the composition by rinsing.

The content of anisic acid or a derivative thereof in the scrubbing composition will generally be at least 1% and will preferably range from 1% to 50%, in particular from 1% to 30% and preferentially from 5% to 20% by weight relative to the total weight of the composition.

This process is more particularly intended for preventing, attenuating and/or combating the signs of ageing of the skin, and especially for attenuating wrinkles and fine lines, and for improving the appearance and/or texture of the skin, in particular for improving the homogeneity of the complexion and/or for smoothing out the skin, and especially for attenuating actinic lentigo or other aesthetic pigmentary disorders (e.g.: brownish spots) or localized hyperpigmentations, or acne or chickenpox marks, and/or for unblocking the skin pores.

The invention will now be illustrated by the non-limiting examples that follow.

### EXAMPLES

### Example 1

### Demonstration of a Desquamating Effect of Anisic Acid

The desquamating properties of anisic acid were demonstrated on a model of skin kept in survival, via analysis of the cohesion of the stratum corneum on histological slices stained with hemalun-eosin.

Fragments of normal human skin are obtained by plastic surgery from 6 different donors. They are placed in inserts, which are themselves positioned on culture wells containing culture medium specifically adapted for maintaining the skin fragments in survival (antibiotics, FCS).

The anisic acid is applied topically according to the following protocol and in comparison with an untreated skin (control skin):
control skin
skin+0.1° anisic acid
skin+% anisic acid

The cohesion of the horny layer was evaluated using stained histological slices, according to the following semi-quantitative scores (evaluation on 10 to 15 fields at magnification 40):
score 0 absence of change of cohesion of the stratum corneum
score 1 slight reduction in cohesion of the stratum corneum
score 2 moderate reduction in cohesion of the stratum corneum
score 3 large reduction in cohesion of the stratum corneum
score 4 very large reduction in cohesion of the stratum corneum with exfoliation.

An average is determined for each parameter from the results obtained on the 6 skin models. The statistical analysis is performed by means of the “reduced deviation” Student test or the paired samples test, with a risk of 5%.

The results are given in the following table:

TABLE
Histological evaluation of the cohesion of the
horny layer after staining with hemalun-eosin (semi-
quantitative scores).

| | | % increase in |
|---|---|---|
| | | desquamation relative |
| Treatment | Scores | to the control skin |
| Control | 0.73 ± 0.2 | 0.0% |

| | | | |
|---|---|---|---|
| Anisic acid | 0.1% | 1.43 ± 0.7* | 95.9% |
| (1) | 1% | 1.57 ± 0.4* | 115% |

(1): para-anisic acid or 4-methoxybenzoic acid or para-methoxybenzoic acid, sold by the company Merck.
*statistically significant difference relative to the control skin (paired Student test, p < 0.05).

Under the experimental conditions of this study, the treatment of the skins with 0.1% and 1% anisic acid shows a significant reduction in the cohesion of the stratum corneum relative to the control skin, with respective scores of 1.43 and 1.57 as opposed to 0.73 for the control skin (the higher the score, the lower the cohesion), which is reflected by an increase in desquamation of +95.9% and +115%, respectively.

These results demonstrate significant desquamating activity after topical application of anisic acid relative to the control skins, which is reflected by a histological reduction in the cohesion of the stratum corneum, without, however, showing a dose effect.

### Example 2

### Formulations

Day cream

| | |
|---|---|
| p-Anisic acid | 0.5% |
| 5-n-Octanoylsalicylic acid | 0.1% |
| Soybean protein | 5% |
| Rice proteiin | 1% |
| Solid fatty substances | 3% |
| Fatty alcohols | 3% |
| Silica | 4% |
| Cyclohexasiloxane | 4% |
| Glycerol | 5% |
| Emulsifiers | 7% |
| Preserving agents | 0.5% |
| Water | qs 100% |

The composition is applied daily to the face for a smoothing effect on the skin's microrelief.

Antisun cream

| | |
|---|---|
| p-Anisic acid | 0.25% |
| Glycerol | 7% |
| Cyclopentasiloxane | 10% |
| Solid fatty substances | 3% |
| Plant oil | 3% |
| Gelling agent | 2% |
| Fatty alcohols | 1% |
| Fatty acids | 3% |
| UV-screening agents | 10% |
| PEG-100 stearate | 2% |
| Preserving agents | 0.5% |
| Alcohol | 5% |
| Water | qs 100% |

This cream is applied to the face in the morning to promote the radiance of the complexion and to protect the skin against the effects of UV.

Makeup-removing cream

| | |
|---|---|
| p-Anisic acid | 0.2% |
| Silica | 1% |
| Ethylhexyl palmitate | 66% |
| Alcohol | 2% |
| Glycerol | 10% |
| Beheneth-10 | 5% |
| Preserving agents | 0.55% |
| Water | qs 100% |

This makeup-removing cream applied to facial skin makes it possible to cleanse the skin gently, to reduce the visibility of the pores and to improve the grain of the skin.

Foaming soap

| | |
|---|---|
| p-Anisic acid | 0.1% |
| Potassium hydroxide | 6% |
| Sorbitol | 7% |
| Fatty substances | 4% |
| Glycerol | 14% |
| Surfactants | 34% |
| Water | qs 100% |

This foaming soap used daily on the skin makes it possible to unblock the pores and to restore the uniform complexion of the skin.

Anti-wrinkle scrubbing composition

| | |
|---|---|
| p-Anisic acid | 5% |
| Glycolic acid | 10% |
| HEPES | 5% |
| Gelling agent | 0.5% |
| Glycerol | 10% |
| Alcohol | 20% |
| Purified water or spring water | qs 100% |

This composition may be applied in the form of a scrub to attenuate facial wrinkles and fine lines, and/or to improve the radiance of the complexion.

The above written description of the invention provides a manner and process of making and using it such that any person skilled in this art is enabled to make and use the same, this enablement being provided in particular for the subject matter of the appended claims, which make up a part of the original description and including the use of a composition comprising, in a physiologically acceptable medium, anisic acid, a salt or a C₁-C₄ alkyl ester thereof, for promoting desquamation of the skin and/or for stimulating epidermal renewal.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A method for promoting desquamation of the skin and/or for stimulating epidermal renewal, comprising applying a composition comprising water and at least one compound selected from the group consisting of anisic acid, a salt of anisic acid, and a C₁-C₄ alkyl ester of anisic acid to the skin of a person in need thereof.

2. A method for:
improving the radiance and/or homogeneity of the complexion and/or reducing the cloudy and/or dull appearance of the complexion;
improving the surface appearance, in particular reducing the rough or cracked appearance of the skin and/or improving the grain and/or softness of the skin;
smoothing out the skin's microrelief, in particular smoothing out the wrinkles and fine lines and/or attenuating acne or chickenpox marks;
attenuating age spots and/or aesthetic pigmentation disorders;
reducing the dryness of the skin, in particular the dryness of aged skin;
improving the appearance and/or reducing the visibility of the pores and/or improving the unblocking of the pores;
promoting the cleansing action and the removal of dead cells at the surface of the skin; and/or
combating the imperfections of greasy skin,
comprising applying a composition comprising water and at least one compound selected from the group consisting of anisic acid, a salt of anisic acid, and a C₁-C₄ alkyl ester of anisic acid to the skin of a person in need thereof.

3. The method according to claim 1, wherein the at least one compound selected from the group consisting of anisic acid, a salt of anisic acid, and a C₁-C₄ alkyl ester of anisic acid is present in the composition in an amount ranging from 0.001% to 50% by weight relative to the total weight of the composition.

4. The method according to claim 2, wherein the at least one compound selected from the group consisting of anisic acid, a salt of anisic acid, and a C₁-C₄ alkyl ester of anisic acid is present in the composition in an amount ranging from 0.001% to 50% by weight relative to the total weight of the composition.

5. The method according to claim 1, wherein the composition further comprises at least one active agent other than anisic acid, a salt of anisic acid, and a C₁-C₄ alkyl ester of anisic acid chosen from desquamating agents, moisturizers, antioxidants, depigmenting agents, agents for stimulating the energy metabolism of cells, agents for stimulating epidermal proliferation or differentiation or for stimulating the synthesis of epidermal or dermal macromolecules, agents for reducing or inhibiting the activity of harmful proteases, agents for promoting the skin's barrier function, tensioning agents, calmatives and anti-seborrhoeic agents, and mixtures thereof.

6. The method according to claim 2, wherein the composition further comprises at least one active agent other than anisic acid, a salt of anisic acid, and a C₁-C₄ alkyl ester of anisic acid chosen from desquamating agents, moisturizers, antioxidants, depigmenting agents, agents for stimulating the energy metabolism of cells, agents for stimulating epidermal proliferation or differentiation or for stimulating the synthesis of epidermal or dermal macromolecules, agents for reducing or inhibiting the activity of harmful proteases, agents for promoting the skin's barrier function, tensioning agents, calmatives and anti-seborrhoeic agents, and mixtures thereof.

7. The method according to claim 5, wherein the other active agent(s) is (are) present in the composition in a content ranging from 0.001% to 10% by weight relative to the total weight of the composition.

8. The method according to claim 6, wherein the other active agent(s) is (are) present in the composition in a content ranging from 0.001% to 10% by weight relative to the total weight of the composition.

9. The method according to claim 1, wherein the composition comprises at least one of para-anisic acid, 4-methoxybenzoic acid and para-methoxybenzoic acid.

10. A composition comprising, in a physiologically acceptable medium, (i) at least one compound selected from the group consisting of anisic acid, a salt of anisic acid, and a C₁-C₄ alkyl ester of anisic acid, and (ii) from 0.001% to 10% by weight, relative to the total weight of the composition, of at least one desquamating agent other than citric acid.

11. The composition according to claim 10, comprising at least one desquamating agent chosen from α-hydroxy acids, β-hydroxy acids, (N-2-hydroxyethylpiperazine-N-2-ethane)sulfonic acid (HEPES); 2-oxothiazolidine-4-carboxylic acid (procysteine) derivatives; urea and derivatives thereof, dioic acid and derivatives thereof.

12. The composition according to claim 10, comprising 5-n-octanoylsalicylic acid.

13. The composition according to claim 10, wherein the at least one compound selected from the group consisting of anisic acid, a salt of anisic acid, and a C₁-C₄ alkyl ester of anisic acid is present in the composition in a content ranging from 0.001% to 10% by weight relative to the total weight of the composition.

14. The process according to claim 1, comprising:
a) topically applying said composition to the skin of the person in need thereof;
b) leaving the composition in contact with the skin for a time of between 5 minutes and 6 hours, and
c) removing the composition by rinsing.

15. The process according to claim 2, comprising:
a) topically applying said composition to the skin of the person in need thereof;
b) leaving the composition in contact with the skin for a time of between 5 minutes and 6 hours, and
c) removing the composition by rinsing.
